(19) United States
(12) Patent Application Publication
(10) Pub. No.: US 2012/0065737 A1
(43) Pub. Date: Mar. 15, 2012

(54) PROSTHETIC FEMORAL STEM FOR USE IN HIGH OFFSET HIP REPLACEMENT

(75) Inventor: James Chow, Paradise Valley, AZ (US)

(73) Assignee: James C. Chow, Paradise Valley, AZ (US)

(21) Appl. No.: 13/227,291

(22) Filed: Sep. 7, 2011

Related U.S. Application Data
(60) Provisional application No. 61/380,396, filed on Sep. 7, 2010.

(51) Int. Cl. A61F 2/32

(52) U.S. Cl. 623/22.42

(57) ABSTRACT

A total hip femoral prosthesis provides high lateral offset with a construct including a conventional length neck. The neck is shifted medially to position the head center in a high offset location. The proximal medial portion of the stem is augmented to provide adequate support to the medialized neck. Modular components are disclosed. Methods of using the prosthesis in total hip arthroplasty are described.

### CROSS-REFERENCE TO RELATED APPLICATIONS

This application claims the benefit of:

U.S. Application No. 61/380,396, filed Sep. 7, 2010, entitled PROSTHETIC FEMORAL STEM FOR USE IN HIGH-OFFSET HIP REPLACEMENT, Attorney's docket no. 165/0001R, which is pending.

The above referenced document is hereby incorporated by reference in its entirety.

### BACKGROUND OF THE INVENTION

This disclosure relates to prostheses and methods for total hip joint replacement, and specifically to high offset hip joint replacement.

Total hip replacement procedures seek to replace a hip joint that has deteriorated in its functionality, range of motion, weight bearing, and most, if not all, other performance and lifestyle attributes. Total hip replacement typically involves amputation of the femoral head, neck, and a portion of the top of the femur in order to replace these structures with prosthetic components.

Individual skeletal development and postures vary from person to person. This is in part due to the three dimensional orientation of the hip socket relative to the proximal femur. The distance between the center of rotation of the femoral head and a reliable anatomical landmark, such as the lesser trochanter, may be described as the vertical offset of the head center from the lesser trochanter. This distance may be measured parallel to the femoral shaft axis, and is relevant to postoperative leg length. In the anterior-posterior view (AP view), the distance between the head center and the shaft axis may be described as the lateral offset of the shaft axis from the head center, or as the medial offset of the head center from the shaft axis. It is often referred to simply as “offset.” Lateral offset may be relevant to postoperative hip abductor function. Lateral offset is independent of the neck-shaft angle. However, lateral offset may be expressed in terms of the neck-shaft angle and the neck length, which is the distance along the neck axis between the head center and the shaft axis or some other reliable landmark. The neck-shaft angle varies through a range of angles, approximately 127-140 degrees for most people. The neck length varies as well. In the lateral view, the distance between the head center and the shaft axis may be described as the anteversion offset of the head center from the shaft axis, or simply anteversion, if the head center is anteriorly displaced from the shaft axis, or as retroversion if the head center is posterior to the shaft axis. Anteversion or retroversion may be relevant to postoperative range of motion.

The neck-shaft angle and/or neck length of a prosthesis can also be highly varied in order to replicate natural anatomy or correct deformity. If the neck-shaft angle and/or neck length of a prosthesis are set so that the lateral offset is comparable to an average lateral offset value for intact normal femora, then this prosthesis may be said to have a conventional or standard offset. However, if the neck-shaft angle and/or neck length are set so that the lateral offset is relatively large, this is referred to as a high offset prosthesis. For example, the neck-shaft angle and/or neck length may be set so that the lateral offset is at least 10 mm greater than the average lateral offset value.

Any of the relevant dimensions of a prosthesis may be set so that they are comparable to an average value for intact normal femora. These dimensions may be said to be conventional dimensions. Likewise, any of the relevant dimensions may be set incrementally greater than the corresponding average value, in which case the prosthetic dimensions may be said to be augmented. It follows that smaller than average dimensions may also be selected.

A surgeon will typically measure both hip joints, including the neck-shaft angles, vertical offset, lateral offset, and leg length, prior to performing a total hip replacement procedure. These measurements allow the surgeon to match the replacement joint as closely as possible to the angles and dimensions of the original hip joint in order to achieve satisfactory range of motion, leg length, soft tissue tension, and stability. These measurements may also allow the surgeon to correct deformity or other conditions in and around the operated joint by matching the replacement joint to the contralateral hip joint.

The development of femoral prostheses with modular necks and heads has allowed the process of fitting the prosthesis to the patient to be both simplified and performed with greater precision. The modular neck is part of a modular prosthesis kit that typically includes an acetabular component, a head component, a neck component, and a stem component. The acetabular component may be a modular construct. Neck components may be made available in a variety of neck lengths and neck-shaft angles. This allows the orthopedic surgeon to select a neck component whose length and angle best approximate the original neck length and angle, so that the lateral offset of the prosthesis approximates that of the original femur. Head components may also contribute to neck length. Head components may be made available with the head-neck connection feature in various positions relative to the head center. For example, a set of heads may be fabricated with internal tapered bores, each bore in the set made progressively deeper in the head. This allows the surgeon to select a head component whose length adds to, or subtracts from, the nominal length of the neck component to further refine the fit.

Profemur® is a trademark for a line of prosthetic joint components and tools owned by Wright Cremascoli Ortho of Milan, Italy. The Cremascoli modular neck was first developed in 1985 and is a precursor to the present technology. The Profemur® modular neck is available in a variety of lengths and angles. For a high offset hip joint replacement, it is standard practice to use a longer length modular neck.

As the neck component becomes longer, it experiences higher stresses due to the longer moment arm between the head center and the neck-stem interconnection or shaft axis. The neck component becomes more prone to failure from stress fractures along the nexus of the neck and stem where stresses are high. Neck component failures require revision surgery to remove the broken neck and any other damaged components and implant replacement components. Revision surgery increases the risk of further complications and may require extensive rehabilitation.

One way to minimize the risk of neck component failure is to make the neck component from a very strong material. However, high strength materials may lack other desirable attributes, such as elasticity or long term in vivo compatibility with head and stem materials.

An alternative approach is to design the components of the modular prosthesis kit to function with a shorter neck component, for example, a neck component whose length is comparable to a conventional length neck component. A shorter neck component should theoretically outlast a longer neck, all else being equal. It is desirable, therefore, to find a solution so that high offset natural hip joints can be replaced with a high offset femoral prosthesis having a more durable conventional or short neck component. Greater durability of the neck and prosthesis reduces the need for potential future surgeries and reduces the overall cost related to future surgery, to the benefit of both the patient and the healthcare system.

This disclosure presents a high offset femoral prosthesis for use in an artificial hip joint system. The femoral prosthesis includes a stem component with an augmented proximal medial portion, and a conventional length modular neck component. The stem component carries the neck component in a medialized location which positions the neck for high offset applications. The augmented region of the stem securely and rigidly supports the conventional modular neck. The overall arrangement reduces the risk of neck failure due to repeated loading and other long term conditions.

In an aspect of the present technology, a prosthetic hip joint system includes a stem component with a distal end adapted for insertion into a prepared canal within the proximal end of the femur. The stem includes a proximal end defining a profile that extends beyond the proximal end of the femur and that includes a mounting socket that receives a distal end of a modular neck component. The mounting socket is medially displaced compared to conventional neck mounting locations. The modular neck has a length and an angular orientation similar to a conventional neck. A head component couples to a proximal end of the modular neck. When assembled, the stem, neck, and head cooperate to position the center of the head at a location defining a high offset geometry. For example, the modular neck defines a center longitudinal axis extending between centers of rotation on each of opposing ends. The neck axis has a length of approximately 26.5 to 38.5 millimeters, which may be described as a conventional or non-high-offset length. Additionally, the neck axis can define an angle with respect to a shaft axis of the stem of between approximately 127 and 143 degrees, which may be described as a conventional or non-high-offset neck angle.

In another aspect, a medical treatment procedure for total hip replacement includes performing an osteotomy to a proximal femur, forming an intramedullary canal for receiving a prosthetic stem, and implanting the stem in the canal so that the proximal end of the stem extends beyond the proximal end of the femur. The proximal end of the stem includes a mounting socket that receives a distal end of a modular neck component. The procedure also includes selecting a modular neck component with a length and an angular orientation of a conventional (non-high-offset) neck, securing the modular neck into the mounting socket, and attaching a head component to a proximal end of the modular neck to as to properly mate with a prepared pelvic socket. The stem, neck, and head cooperate to position the center of the head at a location defining a high offset geometry. This arrangement thereby defines a high offset geometry hip replacement using a conventional non-high-offset modular neck.

In another embodiment, the above described augmented stem design can be used in conjunction with a modular neck that is longer than the conventional length to achieve an ultra-high-offset geometry. This allows accommodation of patients exhibiting such a physiological condition.

### BRIEF DESCRIPTION OF THE DRAWINGS

Various examples of the present technology will now be discussed with reference to the appended drawings. It is appreciated that these drawings depict only typical examples of the invention and are therefore not to be considered limiting of its scope.

FIG. 1 is a front view of a natural proximal femur, with dashed lines indicating anatomical variants with progressively larger lateral offsets;

FIG. 2 is a front view of a prior art total hip femoral prosthesis, with dashed lines indicating a variant having a much larger lateral offset and a slightly larger vertical offset;

FIG. 3 is a front view of another prior art total hip femoral prosthesis, with a cross-hatched area indicating a variant having a ninety degree neck angle, both prostheses having identical lateral and vertical offsets;

FIG. 4 is a front view of yet another prior art total hip femoral prosthesis, with a cross-hatched area indicating a variant having the same lateral offset and a larger vertical offset;

FIG. 5A is a front view of yet another prior art total hip femoral prosthesis, with a stem component, a conventional length neck, and a head component; and FIG. 5B is a front view of the stem and head of FIG. 5A with a longer neck component;

FIG. 6 is a front view of the prior art stem component of FIG. 5A overlaid with two conventional length neck components, one in a conventional position, and the other in a medially offset position;

FIG. 7A is a front view of a stem component according to the present disclosure, with a cross-hatched area indicating proximal medial augmentation to accommodate the medially offset neck component of FIG. 6; and FIG. 7B is a front view of the stem component of FIG. 7A with the medially offset neck component of FIG. 6;

FIG. 8 is a front view of the stem and neck components of FIG. 7B, with dashed lines indicating another neck component with a longer neck length and flatter neck angle in a conventional position, both neck components having identical lateral and vertical offsets;

FIG. 9A is a front view of a total hip femoral prosthesis including the stem and neck components of FIG. 7B with a head component, the prosthesis shown in a cross sectioned proximal femur; and FIG. 9B is a front view of the stem component of FIG. 7B with another neck component with a longer length, the neck component of FIG. 7B shown in dashed lines for comparison;

FIG. 10 is a front view of yet another total hip femoral prosthesis, with a curved proximal lateral profile, shown in a cross-sectioned proximal femur;

FIG. 11 is a front view of yet another total hip femoral prosthesis, with complementary proximal medial and lateral curves, shown in a cross-sectioned proximal femur; and

FIG. 12 is a flowchart of steps in a method of total hip replacement.

### DETAILED DESCRIPTION

The present disclosure sets forth a prosthetic femoral stem for use in a high offset hip replacement procedure.

In this specification, standard medical directional terms are employed with their ordinary and customary meanings. Superior means toward the head. Inferior means away from the head. Anterior means toward the front. Posterior means toward the back. Medial means toward the midline, or plane of bilateral symmetry, of the body. Lateral means away from the midline of the body. Proximal means toward the trunk of the body. Distal means away from the trunk.

In this specification, a standard system of three mutually perpendicular reference planes is employed. A sagittal plane divides a body into bilaterally symmetric right and left portions. A coronal plane divides a body into anterior and posterior portions. A transverse plane divides a body into superior and inferior portions.

In this specification, standard hip anatomical terms are employed with their ordinary and customary meanings.

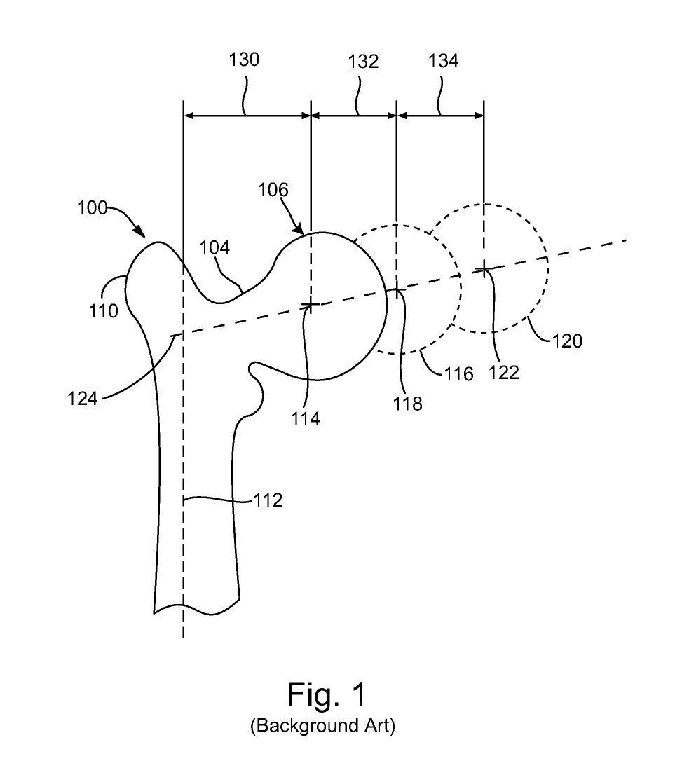

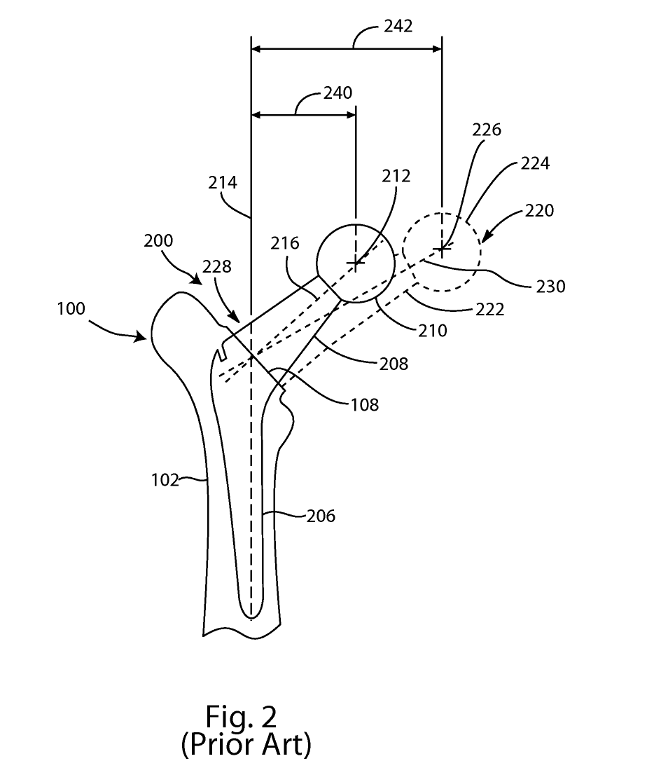

Referring to FIG. 1, an intact natural femur 110 is shown in solid lines in a front view, which may also be described as an anterior-posterior (AP) view. The femur 110 has a proximal end 100 which includes a shaft 102, a neck 104 and a head 106. The shaft 102 has a center longitudinal axis 112. The neck 104 has a center longitudinal axis 124 which is oblique to the stem axis. The head 106 may be approximately spherical. The head has a center point 114, or center of rotation. The center 114 is spaced apart from the shaft axis 112 by a lateral offset distance 130, which is an average lateral offset value, otherwise known as a conventional offset or standard offset.

Lateral offset 130 may be approximately 39.8 mm to 54.2 mm, based on the work of Rubin, et al. as published in 1992 in the British Journal of Bone and Joint Surgery (JBJS BR), volume 74B, pages 28-32, which is incorporated by reference herein in its entirety. Alternately, lateral offset 130 may be 36.2 mm to 49.8 mm, based on the work of Noble, et al. as published in 1988 in Clinical Orthopedics and Related Research (CORR), Number 235, pages 148-165, which is incorporated by reference herein in its entirety.

FIG. 1 also illustrates an anatomical variant of femur 110, which has another approximately spherical femoral head 116 with a center point 118. The center 118 is spaced apart from the shaft axis 112 by a second lateral offset distance 132 which is greater than lateral offset 130. This distance has been exaggerated in FIG. 1 for clarity. This variant may be said to have a high lateral offset. The center 118 may also have a greater vertical offset than center 114 from any reliable anatomical landmark on femur 110. It can be appreciated that the neck 104 in this variant is naturally long enough to reach from the shaft 102 to the head 116.

FIG. 1 also illustrates another anatomical variant of femur 110, which has yet another approximately spherical femoral head 120 with a center 122. The center 122 is spaced apart from the shaft axis 112 by a third lateral offset distance 134 which is greater than the second lateral offset 132. This distance has also been exaggerated in FIG. 1 for clarity. This variant may be said to have a high offset. More specifically, the offset may be characterized as an extra-high offset. The center 122 may also have a greater vertical offset than center 118 from any reliable anatomical landmark on femur 110. It can be appreciated that the neck 104 in this variant is naturally long enough to reach from the shaft 102 to the head 120.

Rubin (JBJS BR 1992) reports offsets up to 62.8 mm. Offsets up to 68.6 mm may be predicted from Rubin's mean of 47.0 mm plus three standard deviations of 7.2 mm. Noble (CORR 1988) reports offsets up to 61.0 mm. Offsets up to 63.4 mm may be predicted from Noble's mean of 43.0 mm plus three standard deviations of 6.8 mm.

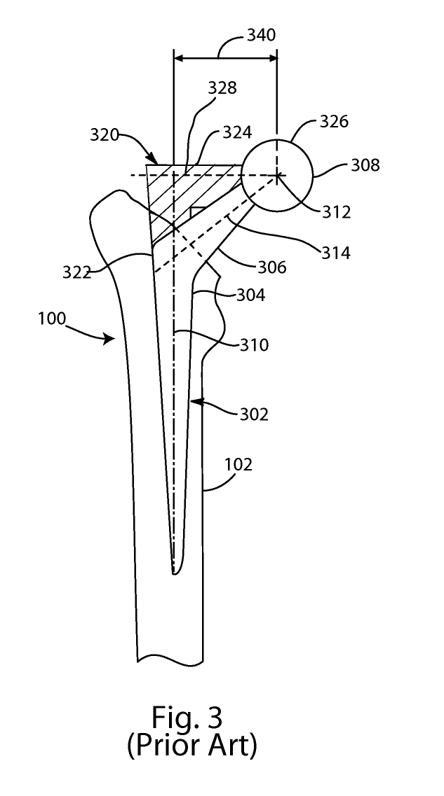

Referring to FIG. 2, a prior art total hip femoral prosthesis 200 is shown implanted in the proximal end 100 of femur 110. Modular femoral prosthesis 200 includes a stem component 206, a neck component 208, and a head component 210. The neck 208 and head 210 and stem 206 are secured together. The stem 206 is shown within a prepared canal in the femoral shaft 102. A femoral neck osteotomy has been performed, removing the neck 104, head 106, and a portion of the proximal end 100, leaving an angled resection surface 108. The stem 206 has a center longitudinal axis 214 which may align with the shaft axis 112 when the stem is implanted in the shaft 102. The neck 208 has a center longitudinal axis 216 which may be oblique to the stem axis 214. The head 210 is spherical and has a center point 212. The center 212 is spaced apart from the stem axis 214 by a lateral offset distance 240, which is a conventional lateral offset.

FIG. 2 also illustrates another prior art total hip femoral prosthesis 220, which includes the stem 206, another neck component 222, and another head component 224. The neck 222 has a center longitudinal axis 230 which may be oblique to the stem axis 214. The head 224 is spherical and has a center point 226. The center 226 is spaced apart from the stem axis 214 by a lateral offset distance 242 which is greater than lateral offset 240. Lateral offset 242 may be called a high offset compared to lateral offset 240. The center 226 may also have a greater vertical offset than center 212 from any reliable anatomical landmark on femur 110. Neck 222 is approximately 38 mm long measured from base to end along the neck axis 230.

It can be appreciated that high offset neck 222 is longer than conventional neck 208. Therefore, neck 222 may experience higher stresses than neck 208 during use. Neck 222 may bend, crack, or break due to high service stresses and/or accidental overload from trips, slips, falls, or other trauma, particularly over years in vivo. Neck 222 may be expected to fail in or near the indicated region 228. Failure of the modular neck 222 will require corrective surgery to replace at least the broken neck, with attendant rehabilitation and risks of significant complications.

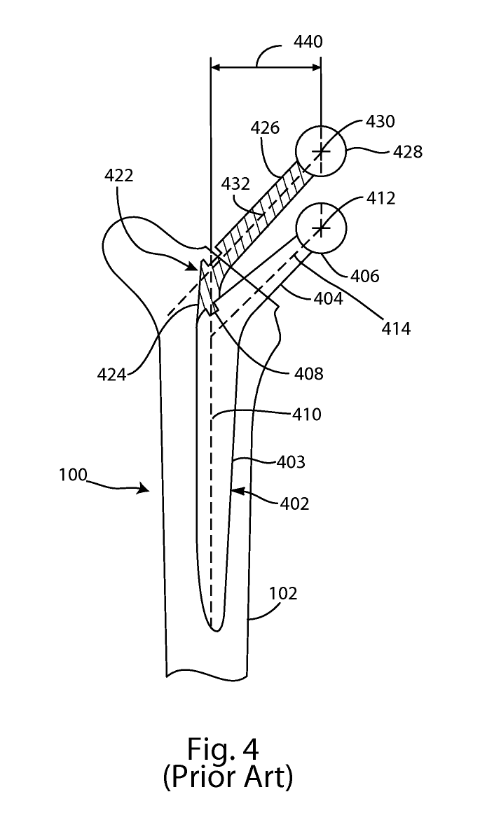

Referring to FIG. 3, a prior art total hip femoral prosthesis 302 is shown implanted in the proximal end 100 of femur 110. Modular femoral prosthesis 302 includes a stem component 304, a neck component 306, and a head component 308. The neck 306 and head 308 and stem 304 are secured together. The stem 304 is shown within a prepared canal in the femoral shaft 102. The femoral shaft 102 has been prepared as set forth above. The stem 304 has a center longitudinal axis 310 which may align with the shaft axis 112 when the stem is implanted in the shaft 102. The neck 306 has a center longitudinal axis 314 which may be oblique to the stem axis 310. The head 308 is spherical and has a center point 312. The center 312 is spaced apart from the stem axis 310 by a lateral offset distance 340.

FIG. 3 also illustrates another prior art total hip femoral prosthesis 320, which includes a longer stem component 322, a horizontal modular neck component 324 and another head component 326. The stem 322 is aligned with stem axis 310. The neck 324 has a center longitudinal axis 328 which may be perpendicular to the stem axis 310, or nearly so. The head 326 is in the same position as head 308. Therefore, head 326 has the same lateral offset 340 and vertical offset as head 308.

It can be appreciated that neck 324 may be shorter than neck 306, however, stem 322 and neck 324 protrude proximally from femur 110 more than stem 304 and neck 306 do. Therefore, stem 322 and neck 324 may hinder abduction by impinging surrounding anatomical structures, such as the lateral pelvis.

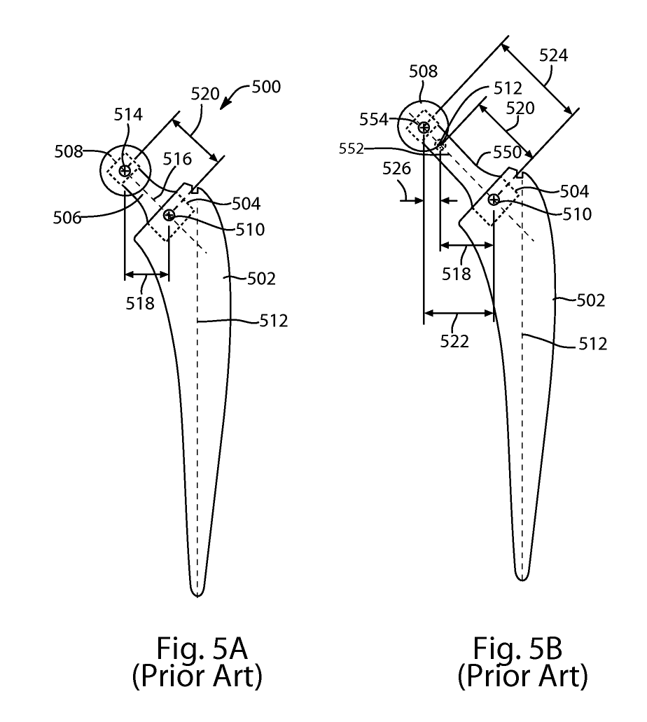

Referring to FIG. 4, a prior art total hip femoral prosthesis 402 is shown implanted in the proximal end 100 of femur 110. Modular femoral prosthesis 402 includes a stem component 403, a neck component 404, and a head component 406. The neck 404 and head 406 and stem 403 are secured together. The stem 403 is shown within a prepared canal in the femoral shaft 102. The femoral shaft 102 has been prepared as set forth above. The stem 403 has a center longitudinal axis 410 which may align with the shaft axis 112 when the stem is implanted in the shaft 102. The stem 403 has a placement slot 408 for a stem inserter instrument (not shown), a tool used for driving the stem into the prepared canal within the femur. The neck 404 has a center longitudinal axis 414 which may be oblique to the stem axis 410. The head 406 is spherical and has a center point 412. The center 412 is spaced apart from the stem axis 410 by a lateral offset distance 440.

FIG. 4 also illustrates another prior art total hip femoral prosthesis 422, which includes another stem component 424, another neck component 426, and another head component 428. The stem 424 is aligned with stem axis 410. The neck 426 has a center longitudinal axis 432 which may be oblique to the stem axis 410. The head 428 is spherical and has a center point 430. The center 430 is spaced apart from the stem axis 410 by lateral offset distance 440. It can be appreciated that center 430 is farther than center 412 from any landmark on femur 110 in a direction parallel to axis 410. Prosthesis 422 may be said to have a high vertical offset compared to prosthesis 402.

Referring to FIG. 5A, a prior art modular total hip femoral prosthesis 500 includes a stem component 502, a neck component 506, and a head component 508. The neck 506 and head 508 and stem 502 are secured together. The stem 502 has a center longitudinal axis 512 which may align with the shaft axis 112 when the stem is implanted in the shaft 102. The stem 502 also has a pocket 504 which receives a distal portion of the neck 506 when the prosthesis 500 is assembled. The neck 506 has a center longitudinal axis 516 which may be oblique to the stem axis 512. A distal center of rotation 510 may be defined on the neck axis 516 in the distal portion of the neck 506. The distal center of rotation 510 may be in the pocket 504 when the neck 506 is secured to the stem 502. The head 508 is spherical and has a center point 514. The center 514 may coincide with the neck axis 516 in a proximal portion of the neck 506 when the head 508 is secured to the neck 506, thus the center 514 may also be referred to as a proximal center of rotation of the neck. The center 514 is spaced apart from the distal center 510 by a medial-lateral distance 518 and a distance 520 along the neck axis 516. Medial-lateral distance 518 is approximately 20 mm to 50 mm. Distance 520 is approximately 20 mm to 30 mm.

Referring to FIG. 5B, a prior art high offset modular total hip femoral prosthesis includes stem 502 and head 508 with a longer modular neck component 550. The neck 550 has a center longitudinal axis 552 which may be oblique to the stem axis 512. The center of head 508 in this arrangement is at point 554. The center 554 is spaced apart from the distal center 510 by a medial-lateral distance 522 and a distance 524 along the neck axis 552. Distance 524 is greater than distance 520. Medial-lateral distance 522 is greater than medial-lateral distance 518 by the increment 526. Increment 526 is approximately 10 mm to 20 mm. Therefore, medial-lateral distance 522 is approximately 30 mm to 70 mm. Distance 524 is approximately 30 mm to 40 mm. Disadvantageously, the increase in lateral offset places significantly greater stress and strain on the longer modular neck 550, as described for neck 222.

Having described various commercially available styles of total hip prostheses in conjunction with long, high offset modular necks, the following description relates to examples of a modular total hip prosthesis which includes an augmented proximal body, and which places the mounting location for a modular neck closer to the hip joint center of rotation, thereby allowing the use of a standard length modular neck. The use of a standard length modular neck may significantly reduce the risk of neck failure as described above.

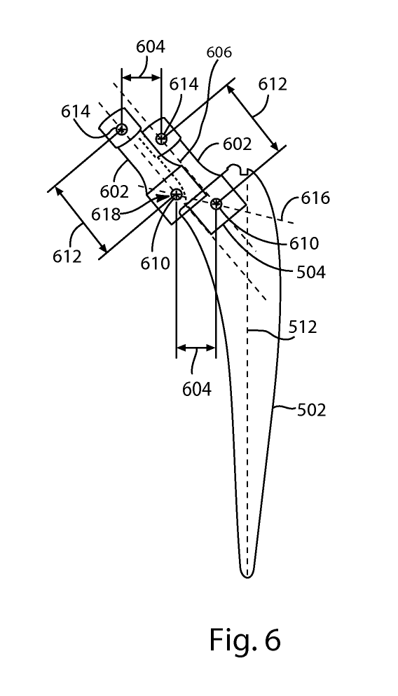

Referring to FIG. 6, stem 502 is shown with a neck component 602. The neck 602 is shown secured in the pocket 504 of the stem 502. The neck 602 has a center longitudinal axis 606 which may be oblique to the stem axis 512. A distal center of rotation 610 may be defined on the neck axis 606 in the distal portion of the neck 602. A proximal center of rotation 614 may be defined on the neck axis 606 in a proximal portion of the neck 602. The proximal center of rotation 614 may correspond to a head center location when a head component (not shown) is attached to neck 602. The proximal center of rotation 614 may correspond to the center of a neutral, or 0 mm head; the center of a head whose length adds to, or subtracts from, the 0 mm datum would be shifted from the proximal center of rotation 614 accordingly. The distal and proximal centers of rotation are spaced apart by a distance 612 along the neck axis 606. Distance 612 is approximately 20 mm to 30 mm.

FIG. 6 also shows a second neck 602 which has been shifted medially by moving the distal center of rotation 610 out of the pocket 504 to location 618 along axis 616. In this example, the second neck 602 has been shifted medially, or medialized, by distance 604 taken perpendicular to stem axis 512. It can be appreciated that the distal center of rotation 610 and the proximal center of rotation of the second neck have both been medialized by distance 604. Distance 604 is approximately 10 mm to 20 mm. Thus, the lateral offset provided by second neck 602 at location 618 is at least 10 mm greater than the lateral offset provided by neck 602 in its original position.

Axis 616 may slope from distal-lateral to proximal-medial, as shown in FIG. 6; from distal-medial to proximal-lateral; parallel to stem axis 512, or perpendicular to stem axis 512. The slope of axis 616 may be selected in order to provide corresponding lateral and vertical offsets during medialization. Medialization along axis 616, sloped as shown, increases lateral offset and vertical offset compared to the starting position in the pocket 504, with greater increase in lateral offset than in vertical offset. A 90 degree slope (parallel to stem axis 512) would increase vertical offset only. A 45 degree slope from distal-lateral to proximal-medial would increase lateral and vertical offset equally. A 0 degree slope (perpendicular to stem axis 512) would increase lateral offset only. A negative slope (from distal-medial to proximal-lateral) would increase lateral offset and decrease vertical offset. The slope of axis 616 may be set based on analysis of measurements from radiographs, CT scans, MRI scans, ultrasound scans, or the like. The slope of axis 616 may be set based on analysis of one or more specific populations, such as a population known to have natural lateral offsets greater than those provided by conventional femoral prosthesis designs, or a population known to have soft tissue laxity or a tendency to dislocate. Data analysis may be expected to reveal trends for vertical offset versus lateral offset in populations such as these.

Modular necks vary in length between 26.5 mm or 27 mm and 38.5 mm, depending on the particular modular neck and based upon conventional, commercially available non-high-offset modular necks.

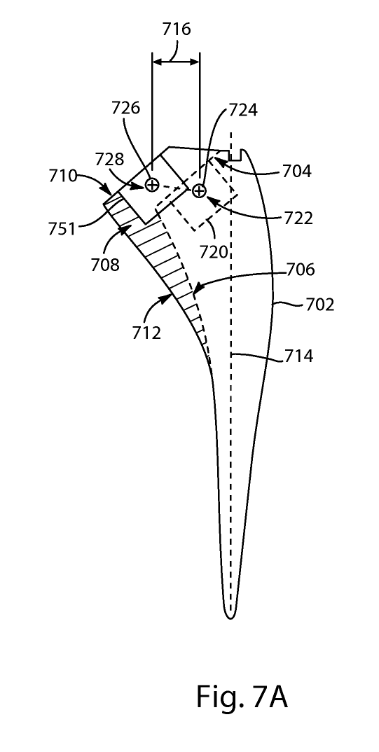

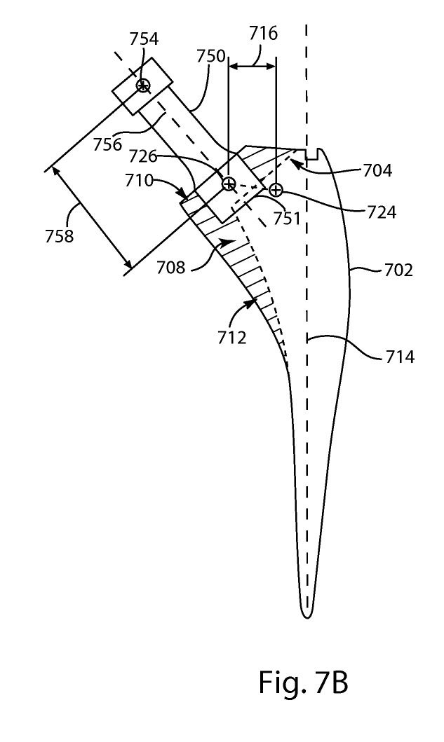

FIG. 7A illustrates how stem 502 may be modified to support the neck 602 with the distal center of rotation 610 in the medialized location 618. A stem component 702 has a center longitudinal axis 714. The stem 702 is shown with a conventionally-positioned pocket 720 to receive a distal portion of a neck component (not shown). A distal center of rotation 724 may be defined in the pocket 720. The distal center of rotation 724 corresponds to the location of a distal center of rotation of a neck component, when assembled to the stem 702. The distal center of rotation 724 is shown in a conventional location 722 comparable to the location of the distal center of rotation 610 in the pocket 504 of stem 502 (FIG. 6). The stem 702 is also shown with a medial curve 706 and an angled proximal surface 704. Proximal surface 704 may correspond to, or align with, resection surface 108 of femur 110 when stem 702 is implanted. Proximal surface 704 may be near, or may coincide with, the distal center of rotation 724. The medial curve 706 and proximal surface 704 may be comparable to a conventional stem, such as stem 502.

The stem 702 is shown with a proximal medial augmented region 708 indicated with cross hatching. The augmented region is integrally formed with the rest of stem 702. The augmented region 708 has a pocket 751, a distal center of rotation 726, a medial curve 712, and an angled proximal surface 710. The pocket 751 and the distal center of rotation 726 are medialized from pocket 720 and distal center of rotation 724 by medial-lateral distance 716 taken perpendicular to stem axis 714. Distance 716 is approximately 10 mm to 20 mm. The distal center of rotation 726 is shown in a medialized location 728, which may be comparable to the medialized location 618 of FIG. 6. The augmented region 708 augments the conventionally sized and shaped portion of stem 702 so that the pocket 751 is surrounded and supported by material. The amount of material added to the proximal medial side of the stem may be equal to distance 716, or may be set so that the pocket 751 is surrounded by an amount of material comparable to that surrounding pocket 720 in a conventional stem, or at least a structural minimum of material. The angled proximal surface 710 may correspond to, or align with, resection surface 108 on the neck 104 of femur 110, or a proximally located resection surface such as a subcapital resection or a mid-neck resection. A proximally located resection may also be described as a high neck osteotomy. Proximal surface 710 may be near, or may coincide with, the distal center of rotation 726.

FIG. 7B illustrates stem 702 with an attached modular neck component 750 anchored in the pocket 751. The modular neck 750 may be secured in the pocket 751 by means of adhesive, cement, wedges, counter wedges, tapers, threading, bolts, friction, or any other biocompatible and structurally appropriate means. The neck 750 has a center longitudinal axis 756 which may be oblique to the stem axis 714. The distal center of rotation 726 is on the neck axis 756 in a distal portion of the neck 750. A proximal center of rotation 754 may be defined on the neck axis 756 in a proximal portion of the neck 750. The distal and proximal centers of rotation are spaced apart by a distance 758 along the neck axis 756. The length 758 of the standard modular neck 750 is approximately 20 mm to 30 mm. The distal center of rotation 726 and the proximal center of rotation 754 are separated by a medial-lateral distance 820 taken perpendicular to stem axis 714. Distance 820 is approximately 20 mm to 50 mm. By medializing the distal center of rotation 726 (and indeed the entire neck 750) compared to a conventional design, the proximal center of rotation 754 may be reached with a standard length neck in a high offset construct, thereby avoiding at least some of the potential failure risks attendant with a longer neck.

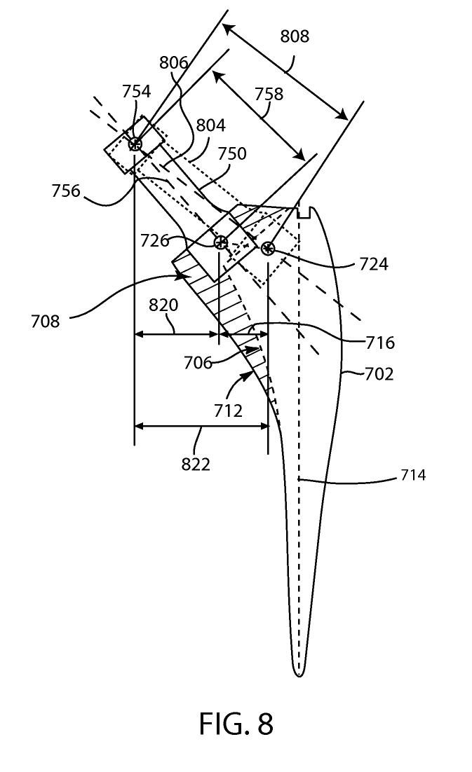

Referring to FIG. 8, the stem 702 and neck 750 of FIG. 7B are compared to a conventional stem with a modular neck component 804 configured to reach from the conventional distal center of rotation 724 to the high offset proximal center of rotation 754. The neck 804 has a center longitudinal axis 806 which may be oblique to the stem axis 714. The distal center of rotation 724 is on the neck axis 806 in a distal portion of the neck 804. The proximal center of rotation 754 is on the neck axis 806 in a proximal portion of the neck 804. The distal and proximal centers of rotation are spaced apart by a distance 808 along the neck axis 806. The length 808 of the longer modular neck 804 is approximately 30 mm to 40 mm. The distal center of rotation 724 and the proximal center of rotation 754 are separated by medial-lateral distance 822 taken perpendicular to stem axis 714. Distance 822 is approximately 30 mm to 70 mm. Distance 820 is less than distance 822 and the reduced length increases the durability of the construct, all else being equal. It is expressly contemplated that the conventional range of sizes and/or orientations in this example and others herein provide for and aid in achieving a custom fit for the hip prosthesis.

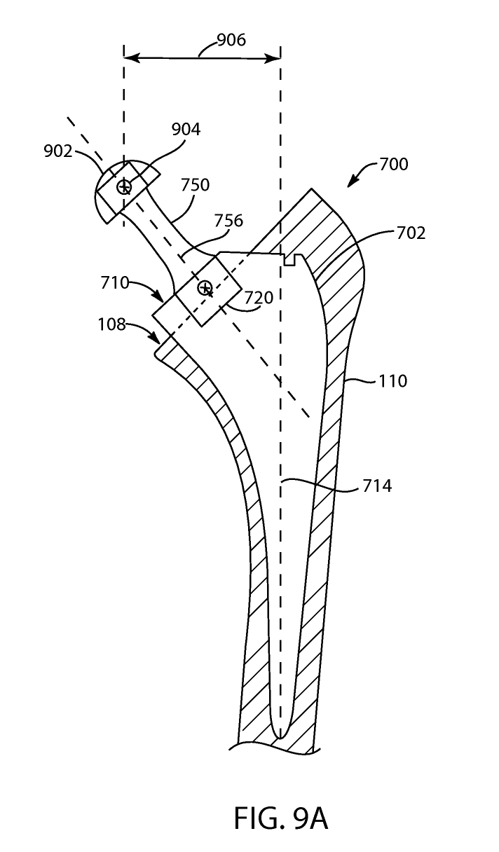

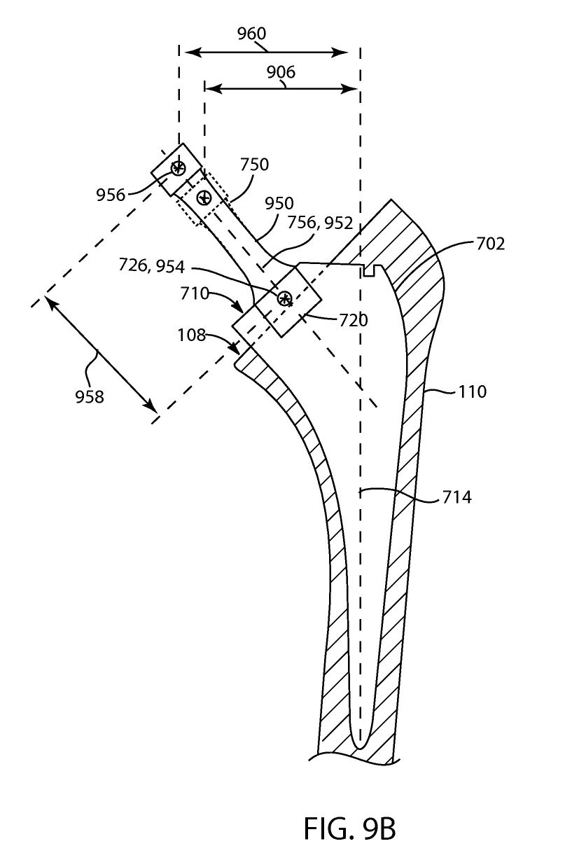

Referring to FIG. 9A, total hip femoral prosthesis 700 is shown implanted in the proximal end 100 of femur 110. Modular femoral prosthesis 700 includes stem 702, neck 750, and a head component 902. The neck 750, head 902, and stem 702 are secured together. A distal portion of the neck 750 is secured in the pocket 720 of the stem 702. The stem 702 is shown within a prepared canal in the femoral shaft 102, as described above. The head 902 is spherical and has a center point 904. The center 904 may coincide with the proximal center of rotation 754 of neck 750. The center 904 is spaced apart from the stem axis 714 by a lateral offset distance 906, which may be at least 10 mm larger than an average lateral offset value for natural femora. Stem 702, neck 750, and head 902 cooperate to provide a high offset head location with a conventional neck length. The proximal surface 710 of the stem 702 protrudes beyond the resection surface 108 of the femur 110. However, the proximal surface 710 may align with a high neck osteotomy.

Referring to FIG. 9B, stem 702 is shown with another modular neck component 950. The neck 950 is secured in the pocket 720 of the stem 702. The neck 950 has a center longitudinal axis 952 which may be oblique to the stem axis 714. A distal center of rotation 954 may be defined on the neck axis 952 in the distal portion of the neck 950. A proximal center of rotation 956 may be defined on the neck axis 952 in a proximal portion of the neck 950. The proximal center of rotation 614 may correspond to a head center location when a head component (not shown) is attached to neck 950. The distal and proximal centers of rotation are spaced apart by a distance 958 along the neck axis 952. The proximal center of rotation 956 is spaced apart from the stem axis 714 by a lateral offset distance 960. The neck 750 of FIGS. 7B-9A is shown in dashed lines for comparison. It can be appreciated that the distal center of rotation 954 coincides with the distal center of rotation 726, the neck axis 952 is collinear with the neck axis 756, and the proximal center of rotation 956 is proximal and medial to the proximal center of rotation 754. The neck length 958 is greater than the neck length 758 and the lateral offset 960 is greater than the lateral offset 906. The stem 702 and the attached neck 950 may be said to form an ultra high offset construct.

This example of an ultra high offset construct is enabled in the present disclosure because the augmented stem 702, when used in combination with modular neck components of various lengths, provides for a greater range of high offset custom fittings than is provided for in a conventional stem with a conventionally positioned neck. In general, it is contemplated that extended modular neck components in a range of lengths of approximately 30 mm to 40 mm can be accommodated in accordance with this embodiment to safely achieve ultra high offset constructs.

It should be clear that a wide range of stem geometries and securing mechanisms can be employed in connection with the high offset stem geometry of this invention. The following are some illustrative geometries to which the principles of this invention have been applied. These are only examples of the range of potential designs contemplated in the scope of this disclosure.

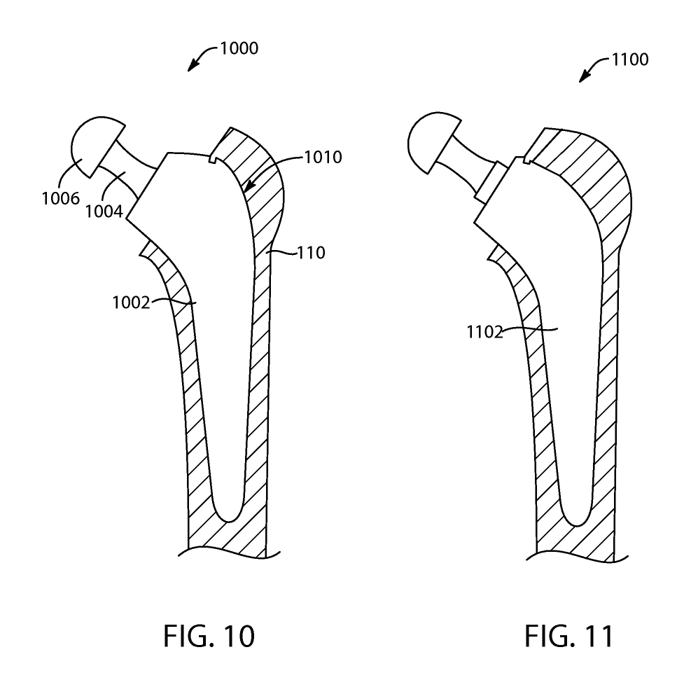

FIG. 10 is an example of a total hip femoral prosthesis 1000 having a stem component 1002 with a proximally medially augmented profile, a modular neck component 1004, and a head component 1006. The prosthesis 1000 is shown within a prepared canal in the femur 110. The prosthetic hip stem 1002 is relatively straight in its distal portion, but as it approaches the neck 1004, it describes a proximal lateral curve 1010. The use of a standard sized neck 1004 in the prosthetic hip system may provide greater durability for this design.

FIG. 11 is an example of a total hip femoral prosthesis 1100 with a stem component 1102 that has a continuously curved proximal profile on both the medial and lateral sides. The stem 1102 may be said to resemble a chili pepper, and is proximally medially augmented to accommodate a short modular neck for greater durability.

Any of the stem components set forth herein may be fabricated from biocompatible materials. Examples of biocompatible materials include, but are not limited to, metals such as stainless steels, titanium and its alloys, cobalt-chrome-molybdenum alloys, and other chromium alloys; polymers such as polyetheretherketone (PEEK) and polyaryletherketone (PAEK); ceramics such as alumina and zirconia; and composites such as carbon fiber reinforced epoxy resin. Portions of a stem component may be fabricated from different materials according to the requirements of each portion. A stem component may be fabricated with a distal portion of one material and a proximal portion of another material. A stem component may be fabricated with a substrate of one material and a coating of another material. The material or materials may be solid (i.e., non-porous) or porous. The surface of a stem component may be smooth or rough on a micro- or macroscopic level. A stem component may be fabricated by casting, forging, machining, or a combination of methods. Coatings may be applied by thermal, chemical, electrical, or comparable means. For example, coatings may be sintered, sputtered, vapor deposited, ion implanted, electroplated, and the like.

Any of the neck components set forth herein may be fabricated as described above for the stem components. Neck components may preferably be fabricated with materials, surface treatments, and methods of fabrication which enhance strength, or minimize unavoidable reductions in strength.

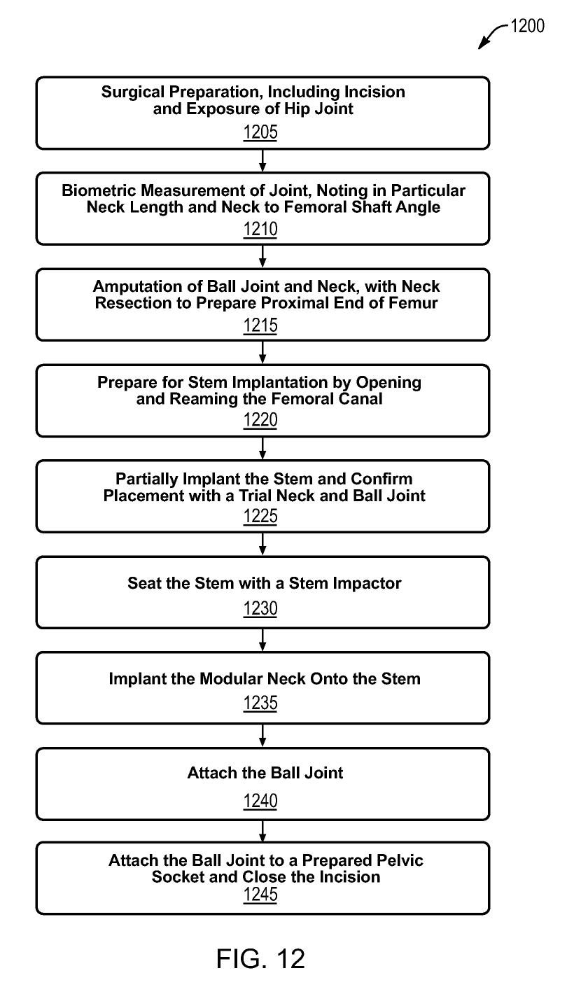

Referring to FIG. 12, an example of a method of total hip replacement 1200 will now be set forth. It is noted that there are no additional steps or techniques required to implant a proximally medially augmented high offset stem. The procedure includes surgical preparation of a natural hip joint by creating an incision and exposing the hip socket (step 1205). The surgeon then performs biometric measurements of the existing hip joint, noting the leg length, neck length, and neck to femur shaft angle (step 1210). The next step is to perform an osteotomy by removing the head, neck and a portion of the proximal femur to create a prepared resection surface (step 1215). The surgeon may prepare the acetabulum and optionally implant at least a portion of an acetabular prosthesis at this point. The surgeon then further prepares the femur by reaming and/or broaching the metaphyseal region and/or medullary canal to define a socket for receiving a prosthetic stem (step 1220). The stem, or a comparable trial stem component, is then implanted but not necessarily firmly seated, and a trial neck and a trial head are placed in the socket to confirm the placement of the stem (step 1225). A trial acetabular liner or articular insert may also be used in this step. The prosthetic stem is then firmly tamped into the canal with a stem impactor (step 1230), so that the proximal end of the stem extends beyond the proximal end of the femur. The proximal end of the stem includes a mounting socket that receives a distal end of a modular neck component, which is implanted so as to locate a head component on a proximal end of the modular neck at a location defining a high offset geometry. Based upon the prior biometrics and placement of the trial neck and head, a modular neck component is selected so that the neck defines a length and an angular orientation of a conventional, non-high-offset neck, and that conforms to the geometry of the replaced natural hip joint, or a corrected version thereof. The modular neck is secured into the mounting socket (step 1235). A head component is attached to a proximal end of the modular neck to as to properly mate with a prepared acetabular socket (step 1240). The femur, with the prosthetic head, neck and stem, is now positioned so that the head engages the prepared acetabular socket and the incision is closed (step 1245). This arrangement thereby defines a high offset geometry hip replacement using a conventional non-high-offset modular neck.

The foregoing has been a detailed description of illustrative embodiments of the invention. Various modifications and additions can be made without departing from the spirit and scope of this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. For example, the distance between the proximal end of the prosthetic stem and the proximal end of the femur can be increased or decreased, to meet the specific biometric proportions of the patient. The augmentation of the curved profile of the stem below the proximal end can be increased or decreased based on necessary accommodation to the load and stress on the distal end of the modular neck. The proximal end of the prosthetic stem can be fashioned to as to accommodate a plurality of proximal ends of different heights, so as to allow custom fitting at the time of implantation. Accordingly, this description is meant to be taken only by way of example, and not to otherwise limit the scope of this invention.

The present technology may be embodied in other specific forms without departing from its spirit or essential characteristics. It is appreciated that various features of the above described examples can be mixed and matched to form a variety of other alternatives. As such, the described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A femoral prosthesis for total hip replacement, comprising:
a stem, wherein the stem comprises a center longitudinal stem axis;
a neck coupled to the stem, wherein the neck comprises a center longitudinal neck axis, a distal center of rotation on the neck axis in a distal portion of the neck, and a proximal center of rotation on the neck axis in a proximal portion of the neck, wherein the distance between the distal and proximal centers of rotation along the neck axis is between 26.5 mm and 38.5 mm, wherein the distal portion of the neck is coupled to a proximal medial portion of the stem; and
a spherical head coupled to the proximal portion of the neck, wherein the head component comprises a head center of rotation;
wherein the stem, neck, and head are coupled together so that the head center of rotation is at least 30 mm from the stem axis.

2. The femoral prosthesis of claim 1, wherein the head center of rotation is at least 50 mm from the stem axis.

3. The femoral prosthesis of claim 1, wherein the distance between the distal and proximal centers of rotation perpendicular to the stem axis is between 20 mm and 50 mm.

4. The femoral prosthesis of claim 1, wherein a proximal lateral profile of the stem is convex.

5. The femoral prosthesis of claim 4, wherein the stem has a continuously curved proximal profile on both the lateral profile and a medial and a medial profile.

6. The femoral prosthesis of claim 1, wherein the stem and neck are coupled together by integral formation.

7. The femoral prosthesis of claim 1, wherein the stem and neck are separate components.

8. A femoral prosthesis for total hip replacement, comprising:
a stem, wherein the stem comprises a center longitudinal stem axis;
a neck coupled to the stem, wherein the neck comprises a center longitudinal neck axis, a distal center of rotation on the neck axis in a distal portion of the neck, and a proximal center of rotation on the neck axis in a proximal portion of the neck, wherein the distance between the distal and proximal centers of rotation perpendicular to the stem axis is between 20 mm and 50 mm, wherein the distal portion of the neck extends from a proximal medial portion of the stem; and
a spherical head coupled to the proximal portion of the neck, wherein the head component comprises a head center of rotation;
wherein the stem, neck, and head are coupled together so that the head center of rotation is at least 30 mm from the stem axis.

9. The femoral prosthesis of claim 8, wherein the head center of rotation is at least 50 mm from the stem axis.

10. The femoral prosthesis of claim 8, wherein the distance between the distal and proximal centers of rotation along the neck axis is between 26.5 mm and 38.5 mm.

11. The femoral prosthesis of claim 8, wherein a proximal lateral profile of the stem is convex.

12. The femoral prosthesis of claim 11, wherein the stem has a continuously curved proximal profile on both the lateral profile and a medial and a medial profile.

13. The femoral prosthesis of claim 8, wherein the stem and neck are coupled together by integral formation.

14. The femoral prosthesis of claim 8, wherein the stem and neck are separate components.

15. A femoral prosthesis for total hip replacement, comprising:
a stem, wherein the stem comprises a center longitudinal stem axis and a pocket, wherein the pocket is recessed into a proximal medial portion of the stem;
a neck coupled to the stem, wherein the neck comprises a center longitudinal neck axis, a distal center of rotation on the neck axis in a distal portion of the neck, and a proximal center of rotation on the neck axis in a proximal portion of the neck, wherein the distance between the distal and proximal centers of rotation along the neck axis is between 26.5 mm and 38.5 mm, wherein the distal portion of the neck is received in the pocket; and
a spherical head coupled to the proximal portion of the neck, wherein the head component comprises a head center of rotation;
wherein the stem, neck, and head are coupled together so that the head center of rotation is at least 30 mm from the stem axis.

16. The femoral prosthesis of claim 15, wherein the head center of rotation is at least 50 mm from the stem axis.

17. The femoral prosthesis of claim 15, wherein the distance between the distal and proximal centers of rotation perpendicular to the stem axis is between 20 mm and 50 mm.

18. The femoral prosthesis of claim 15, wherein a proximal lateral profile of the stem is convex.

19. The femoral prosthesis of claim 18, wherein the stem has a continuously curved proximal profile on both the lateral profile and a medial and a medial profile.

20. The femoral prosthesis of claim 15, wherein the stem and neck are separate components.

21. A prosthetic hip joint system comprising:
a stem having a distal end adapted for insertion a prepared canal within the proximal end of the femur, the stem including a proximal end defining a profile that extends beyond the proximal end of the femur and that includes a mounting socket that receives a distal end of a modular neck, so as to locate a ball joint on a proximal end of the modular neck at a location defining a high-offset geometry; and
wherein the modular neck defines a length and an angular orientation of a non-high-offset neck.

22. The prosthetic hip joint system as set forth in claim 21 wherein the modular neck defines a medial axis having a length of between centers of rotation on each of opposing ends of approximately 26.5 to 38.5 millimeters.

23. The prosthetic hip joint system as set forth in claim 21 wherein the modular neck defines a medial axis having an angle with respect to an axis of the femur of between approximately 127 and 143 degrees.

24. The prosthetic hip joint system as set forth in claim 21 wherein the proximal end of the stem includes a mounting socket that enables attachment of the modular neck subsequent to implantation of the stem into the femur.

25. A medical treatment procedure for total hip replacement comprising the steps of:
performing an osteotomy to a femur and defining a canal for receiving a prosthetic stem;
implanting a stem in the canal, the stem defining a distal end adapted for insertion a prepared canal within the proximal end of the femur, the stem including a proximal end defining a profile that extends beyond the proximal end of the femur and that includes a mounting socket that receives a distal end of a modular neck, so as to locate a ball joint on a proximal end of the modular neck at a location defining a high-offset geometry;
selecting a modular neck, wherein the modular neck defines a length and an angular orientation of a non-high-offset neck, and securing the modular neck into the mounting socket; and
attaching a ball joint to a proximal end of the modular neck to as to properly mate with a prepared pelvic socket in a high-offset geometry.
